(19) United States
(12) Patent Application Publication
(10) Pub. No.: US 2026/0308606 A1
(43) Pub. Date: Oct. 8, 2026

(54) METHOD OF PRODUCING CURCUMINOIDS

(71) Applicant: NICOVENTURES TRADING LIMITED, London (GB)

(72) Inventors: Omer BAYAZEID, Winchester (GB); Anupriya THIND, London (GB); Sara BEN KHALED, Southampton (GB)

(21) Appl. No.: 19/479,146

(22) PCT Filed: Apr. 26, 2024
(86) PCT No.: PCT/GB2024/051091

§ 371 Date: Oct. 27, 2025

(30) Foreign Application Priority Data
Apr. 28, 2023 (GB) 2306347.2

(51) Int. Cl. C12N 15/82; C12N 9/10

(52) U.S. Cl. CPC C12N 15/8243; C12N 9/1029

(57) ABSTRACT

The present disclosure relates to a composition, cells transformed with the composition, use of the composition, methods of using the composition, and products obtainable from the method.

### FIELD OF THE INVENTION

The present disclosure relates to a composition, cells transformed with the composition, use of the composition, methods of using the composition, and products obtainable from the method.

### BACKGROUND OF THE INVENTION

In 2021, the global dietary supplements market size was valued at USD 151.9 billion, with an expected compound annual growth rate of 8.9% from 2022 to 2030 (Grand View Research. Report ID: 978-1-68038-919-7. Retrieved from https://www.grandviewresearch.com/industry-analysis/dietary-supplements-market). Of this, the global curcumin market size was valued at USD 73.41 million with an expected compound annual growth rate of 9.95% between 2022 and 2029 (Fortune Business Insights. Report ID: FBI101453. Retrieved from https://www.fortunebusinessinsights.com/industry-reports/curcumin-market-101453).

Curcumin is a natural compound derived from the spice turmeric, which has received interest from both the medical/scientific and culinary worlds. Turmeric is extracted from the rhizome of Curcuma longa (Zingiberaceae), a herbaceous perennial plant which is part of the ginger family. Curcuma longa has been, and still is, used as a medicinal herb, predominantly in Asian countries, where the plant originates. Curcuma longa, in particular curcumin derived from it, is of interest, as it has bioactive properties which include antioxidant, anti-inflammatory, antimutagenic, antimicrobial and anticancer effects. Therefore, turmeric and curcumin not only have use as a traditional spice in the food and beverage industries, but have also been incorporated into cosmetics, colorants, dietary supplements and medical products, such as antiseptics (Hewlings and Kalman. Curcumin: A review of its effects on human health. Foods. 2017, 6, 92).

Curcumin is a curcuminoid, which are a small class of plant secondary metabolites. Curcuminoids also comprise the active compounds demethoxycurcumin and bisdemethoxycurcumin and other curcuminoid derivatives such as 5′-methoxycurcumin, dihydrocurcumin, and cyclocurcumin Sandur et al. Curcumin, demethoxycurcumin, bisdemethoxycurcumin, tetrahydrocurcumin and turmerones differentially regulate anti-inflammatory and anti-proliferative responses through a ROS-independent mechanism. Carcinogenesis, 2007. 28, 8, 1765-1773; Omosa et al. 19.3—Chemistry of Curcuma longa, Ed: Kuete, V. Medicinal Spices and Vegetables from Africa, Elsevier, 2017, 425-435). Curcuminoids contain phenolic hydroxyl groups, a heptadiene chain, and a diketone moiety, which contribute to their diverse therapeutic properties, including antioxidant, anti-inflammatory, and anticancer effects. Whilst curcumin has the highest antioxidant activity of the curcuminoids, curcumin, demethoxycurcumin and bisdemethoxycurcumin, it is also the least stable, with bisdemethoxycurcumin followed by demethoxycurcumin less susceptible to degradation (Mukherjee & Kar. 6-Curcuminoids: The Novel Molecules of Nature, Ed: Ahmad, R. B. Herbs and Spices-New Processing Technologies, IntechOpen, 2021. Retrieved from https://www.intechopen.com/chapters/77905). Therefore, curcuminoids as a class present compounds of interest for commercial utilisation.

### SUMMARY OF THE INVENTION

In one aspect, there is provided a composition for promoting the expression of one or more curcuminoids in a cell, wherein the composition comprises:
a) one or more first vector components encoding one or more enzymes present in the curcuminoid pathway; and optionally
b) one or more second vector components encoding one or more enzymes present in the curcuminoid pathway, different to the one or more enzymes present in the curcuminoid pathway encoded by the first vector component.

In one embodiment, the one or more first vector components are provided in a first vector and the one or more second vector components are provided in a second vector.

In one embodiment, the one or more first vector components and the one or more second vector components are provided in a single vector.

In one aspect, there is provided a plant, plant part or plant cell transformed with the vector components according to any one of the composition embodiments described herein.

In one aspect, there is provided use of the composition according to any one of the composition embodiments described herein for promoting the expression of one or more curcuminoids in algae, moss, or any plant, plant part, or plant cells.

In one aspect, there is provided a method of promoting the expression of one or more curcuminoids in a cell, comprising the step of:
a) introducing the composition according to any one of the composition embodiments described herein into a cell.

Optionally, the method further comprises the steps of:
b) extracting the one or more curcuminoids from the cell to produce an extract;
c) detecting the one or more curcuminoids in the extract; and
d) purifying the one or more curcuminoids from the extract.

In one aspect, there is provided a curcuminoid obtainable from the method according to any one of the method embodiments described herein.

### BRIEF DESCRIPTION OF THE DRAWINGS

Embodiments of the invention will now be described, by way of example only, with reference to accompanying drawings, in which:

FIG. 1 shows HPTLC results for Nicotiana tabacum leaves transfected with or without the composition according to the invention, wherein the enzymes encoded by the vector are DCS and a CURS. Curcumin denotes the curcumin standard, 71 denotes leaves transfected with DCS and CURS3 genes, T2 denotes leaves transfected with DCS, CURS3 and DEL/ROS genes, and T3 denotes leaves transfected with DCS, CURS3 and MYB12 genes. Stationary Phase; Merck, HPTLC Silica gel 60 F₂₅₄, Mobile phase; dichloromethane-methanol 9.7:0.3 (v/v). All samples were dissolved in Methanol.

FIG. 2 schematically shows a customized pCAmbia2300 vector encoding DCS and CURS3 for plant cell lines and Nicotiana species.

FIG. 3 schematically shows a customized pUC-GW vector encoding DCS and CURS3 for algae.

FIGS. 4A-D schematically show the biosynthetic pathway for curcumin and curcumin glucosides. Enzymatic activity is indicated by arrows. FIG. 4A shows the phenylpropanoid pathway which provides the precursor compounds for curcuminoid synthesis. FIG. 4B shows the conversion of feruloyl-CoA (a phenylpropanoid) to curcumin and demethoxycurcumin. FIG. 4C shows the conversion of P-coumaroyl-CoA (a phenylpropanoid) to demethoxycurcumin and bisdemethoxycurcumin. FIG. 4D shows the conversion of curcumin to various curcumin glucoside compounds.

### BRIEF DESCRIPTION OF THE SEQUENCES

A summary of sequence identifiers used throughout the subject specification and the corresponding sequence listing is provided wherein:

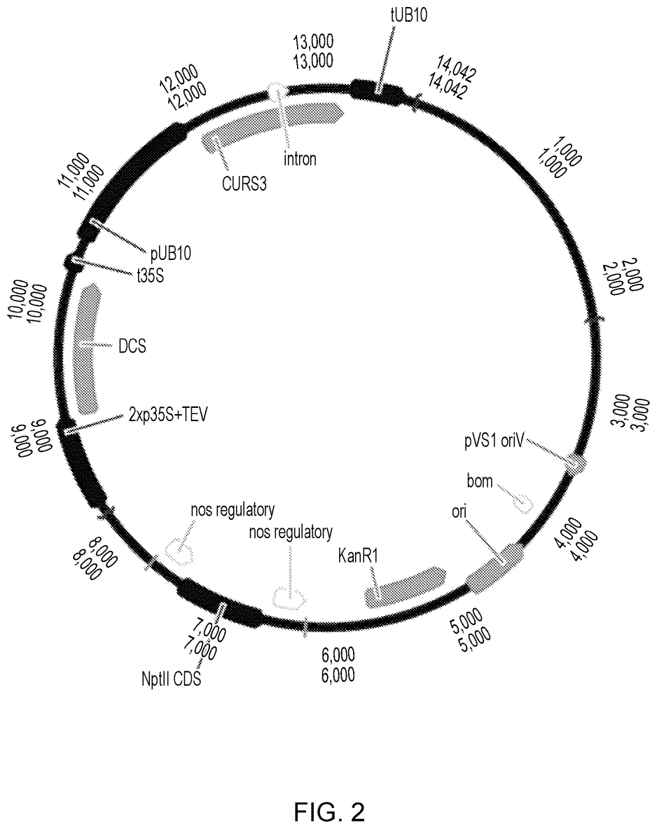

SEQ ID NO. 1 corresponds to the nucleic acid sequence of the pCAmbia2300 vector of FIG. 2, which encodes DCS and CURS3.

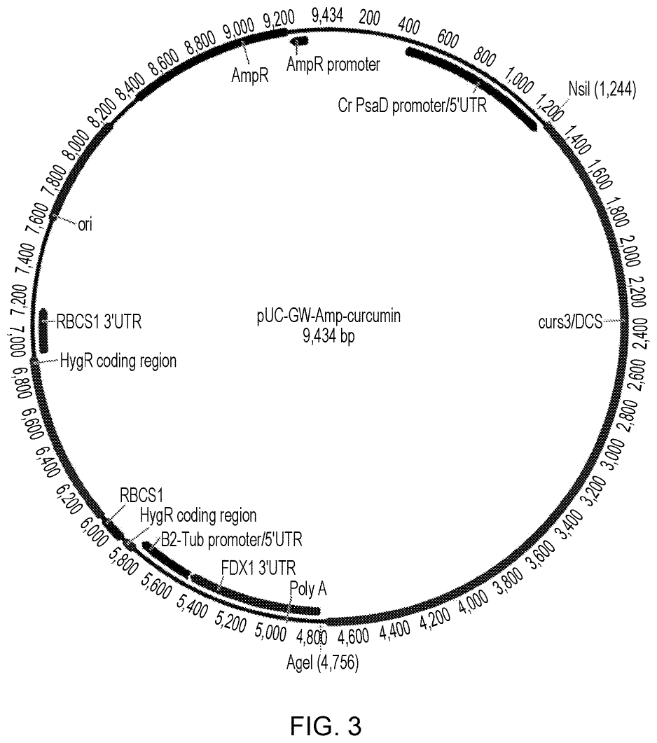

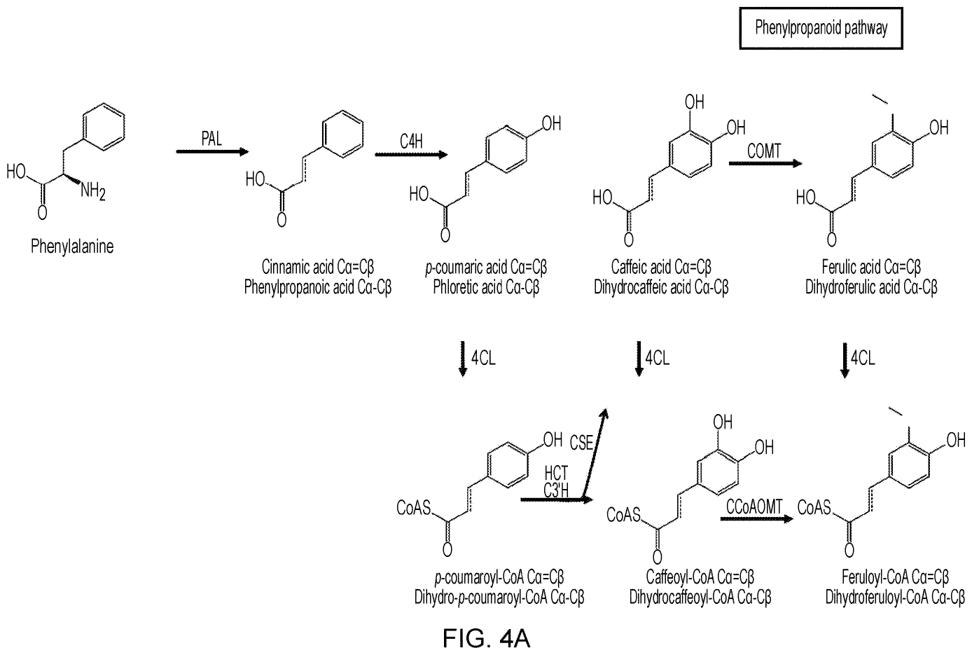

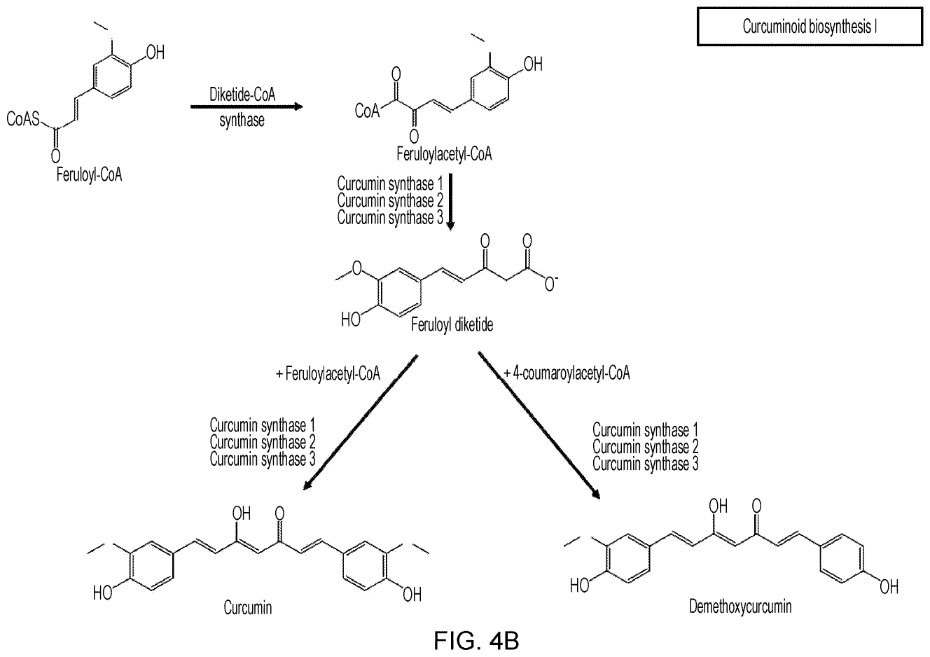

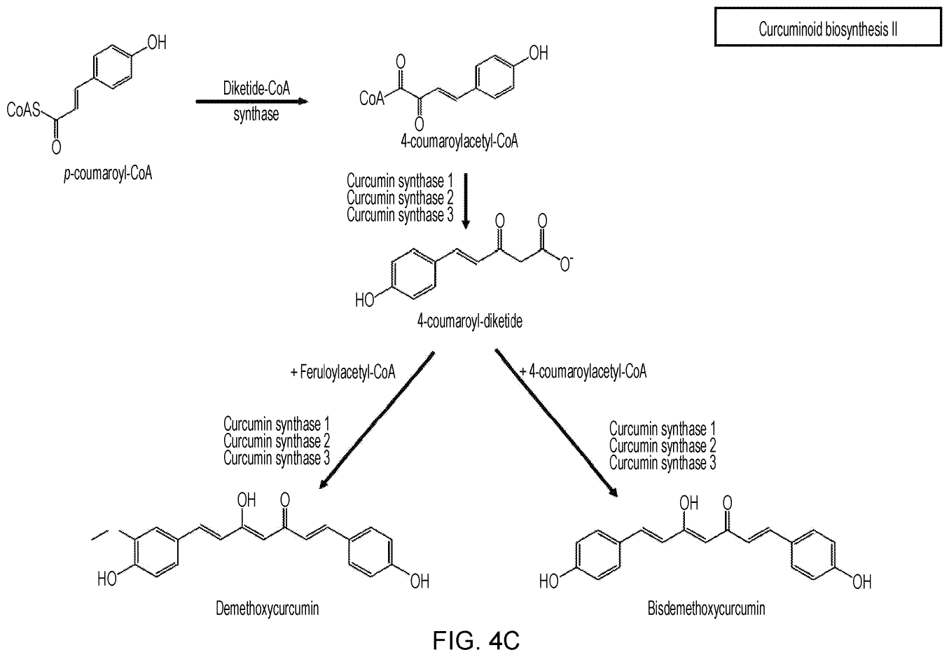

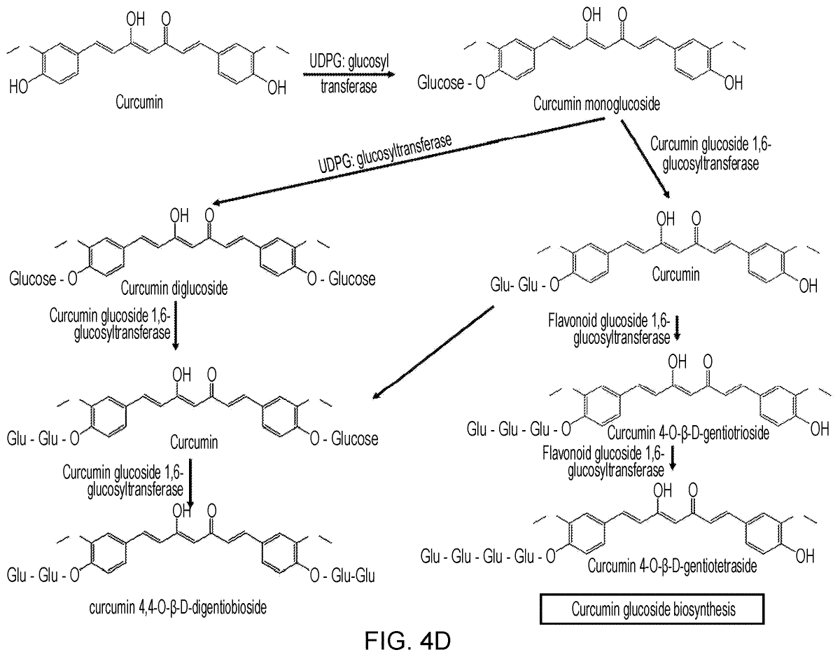

SEQ ID NO. 2 corresponds to the nucleic acid sequence of the pUC-GW vector of FIG. 3, which encodes DCS and CURS3.

### Definitions

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. Accordingly, the terms defined immediately below are more fully defined by reference to the specification as a whole.

Where “enzymes present in the curcuminoid pathway” are disclosed, the skilled person will understand that the curcuminoid pathway may encompass all of the pathway from the phenylalanine starting point through to a curcuminoid end product. The curcuminoid pathway may be divided into two subdivisions. The first stage being the phenylpropanoid synthesis pathway, the second stage being curcuminoid biosynthesis.

It is to be understood that a polypeptide (synonymous with “protein” and/or “enzyme”) may be coded for by more than one nucleotide sequence due to the degeneracy of the genetic code. Therefore, a polynucleotide encoding the vector components of the invention may have at least 70%, 75%, 80%, 85%, 90%, 95%, 96%, 97%, 98% or 99% sequence identity to the disclosed nucleotide sequences (SEQ ID NO: 1 or 2).

Other definitions of terms may appear throughout the specification. Before the exemplary aspects and embodiments are described in more detail, it is to understand that this disclosure is not limited to particular aspects or embodiments described, as such may, of course, vary. It is also to be understood that the terminology used herein is for the purpose of describing particular aspects or embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an enzyme” or “a curcuminoid” includes a plurality of such candidate agents and equivalents thereof known to those skilled in the art, and so forth.

### DETAILED DESCRIPTION OF THE INVENTION

Curcuminoids present a small family of compounds with a number of bioactive properties and commercial uses. Therefore, it would be advantageous to develop methods to promote the expression of one or more curcuminoids, such that they can be extracted for further use.

In one aspect, there is provided a composition for promoting the expression of one or more curcuminoids in a cell, wherein the composition comprises:
a) one or more first vector components encoding one or more enzymes present in the curcuminoid pathway; and optionally
b) one or more second vector components encoding one or more enzymes present in the curcuminoid pathway, different to the one or more enzymes present in the curcuminoid pathway encoded by the first vector component.

In one embodiment, the one or more first vector components are provided in a first vector and the one or more second vector components are provided in a second vector.

In one embodiment, the one or more first vector components and the one or more second vector components are provided in a single vector.

The skilled person will understand that the number of vectors which comprise the vector components of the composition, will depend on the enzymes present in the curcuminoid pathway that they wish to introduce to a cell. The claimed composition provides flexibility to tailor the encoded vector components to the suit the desired promotion of curcuminoids.

It will be understood that in some embodiments, the one or more first vector components may be present in a single vector. In other embodiments, the one or more first vector components may be present in more than one vector, such as two, three, four, five, six or more vectors. It will be understood that in some embodiments, the one or more second vector components may be present in a single vector. In other embodiments the one or more second vector components may be present in more than one vector, such as two, three, four, five, six or more vectors.

The number of vectors used for each vector component is independently selected such that the one or more first vector components may be present in a single vector whilst the one or more second vector components may be present in more than one vector, and vice versa.

It will also be understood that the invention contemplates all combinations of the vector components disclosed herein. For example, the invention contemplates a first vector comprising one or more first vector components, a second vector comprising one or more second vector components and a third vector comprising one or more first vector components and one or more second vector components.

In one embodiment, the total nucleotide sequence length encoded by the first and/or second vector components is 200 KB or less.

In one embodiment, the total nucleotide sequence length encoded by the first and/or second vector components is 190 KB or less.

In one embodiment, the total nucleotide sequence length encoded by the first and/or second vector components is 180 KB or less.

In one embodiment, the total nucleotide sequence length encoded by the first and/or second vector components is 170 KB or less.

In one embodiment, the total nucleotide sequence length encoded by the first and/or second vector components is 160 KB or less.

In one embodiment, the total nucleotide sequence length encoded by the first and/or second vector components is 150 KB or less.

In one embodiment, the total nucleotide sequence length encoded by the first and/or second vector components is 140 KB, 130 KB, 120 KB, 110 KB, 100 KB, 90 KB, 80 KB, 70 KB, 60 KB, 50 KB, 40 KB, 30 KB, 20 KB, 10 KB or less.

In one embodiment, the total nucleotide sequence length encoded by the first and/or second vector components is 10 KB or more.

In one embodiment, the total nucleotide sequence length encoded by the first and/or second vector components is 20 KB or more.

In one embodiment, the total nucleotide sequence length encoded by the first and/or second vector components is 30 KB or more.

In one embodiment, the total nucleotide sequence length encoded by the first and/or second vector components is 40 KB or more.

In one embodiment, the total nucleotide sequence length encoded by the first and/or second vector components is 50 KB or more.

In one embodiment, the total nucleotide sequence length encoded by the first and/or second vector components is 60 KB, 70 KB, 80 KB, 90 KB, 100 KB, 110 KB, 120 KB, 130 KB, 140 KB, 150 KB, 160 KB, 170 KB, 180 KB, 190 KB, 200 KB or more.

In one embodiment, the total nucleotide sequence length encoded by the first and/or second vector components is between 100 KB and 150 KB.

The total nucleotide sequence length of the first and/or second vector components will depend on whether they are to be inserted independently into two or more vectors, or in combination in a single vector. The skilled person will understand that the total nucleotide sequence length of the vector components is dependent on the vector construct to which they are to be inserted and thus could be greater than 200 KB in certain circumstances.

The skilled person will also understand that the vector construct in which the first vector component and/or second vector component are contained will be selected based on the skilled persons knowledge of suitable vectors for the species of interest. The vector constructs disclosed herein are examples of suitable constructs for algae, moss and plant species. However, other vector constructs may also be appropriate and the skilled person would be aware how to select an appropriate vector construct for their intended species.

Therefore, the skilled person will use their knowledge and the information disclosed herein to determine the total sequence length and the vector construct required for efficient transformation into the species of interest.

In one embodiment, one or more vectors comprising the first vector component and/or second vector component are added to a cell.

In one embodiment, two or more vectors comprising the first vector component and/or second vector component are added to a cell.

In one embodiment, three or more vectors comprising the first vector component and/or second vector component are added to a cell.

In one embodiment, four or more vectors comprising the first vector component and/or second vector component are added to a cell.

In one embodiment, five or more vectors comprising the first vector component and/or second vector component are added to a cell.

In one embodiment, six or more vectors comprising the fi first vector component and/or second vector component are added to a cell.

In one embodiment, seven or more vectors comprising the first vector component and/or second vector component are added to a cell.

In one embodiment, eight or more vectors comprising the first vector component and/or second vector component are added to a cell.

In one embodiment, nine or more vectors comprising the first vector component and/or second vector component are added to a cell.

In one embodiment, ten or more vectors comprising the first vector component and/or second vector component are added to a cell.

In one embodiment, up to ten vectors comprising the first vector component and/or second vector component are added to a cell.

In one embodiment, the cell is a plant cell.

In a further embodiment, the plant cell is an algae cell, moss cell, or any cell or cell line derived from the genus Nicotiana.

In an alternative embodiment, the cell is a yeast cell or a bacterial cell.

In yet a further embodiment, the cell or cell line derived from the genus Nicotiana is selected from the group comprising or consisting of Nicotiana tabacum, Nicotiana benthamiana, or BY-2 cells.

The skilled person will understand that the type of vector selected will depend on its intended purpose. The vector may be any suitable type of vector such as a plasmid, cosmid, phage, or viral vector.

In one embodiment, the vector comprising the first vector component and/or second vector component is a plasmid vector.

In a further embodiment, the plasmid vector is a plasmid binary vector.

For ease of reference, these and further aspects of the present invention are now discussed under appropriate section headings. However, the teachings under each section are not necessarily limited to each particular section.

### Enzymes Present in the Curcuminoid Pathway

As described herein, the composition for promoting the expression of one or more curcuminoids comprises one or more first vector components encoding one or more enzymes present in the curcuminoid pathway, and optionally one or more second vector components encoding one or more enzymes present in the curcuminoid pathway, different to the one or more enzymes present in the curcuminoid pathway encoded by the first vector component.

In one embodiment, the one or more enzymes present in the curcuminoid pathway comprise DCS.

In one embodiment, the one or more enzymes present in the curcuminoid pathway comprise a CURS.

In one embodiment the one or more enzymes present in the curcuminoid pathway are DCS and a CURS.

In one embodiment, the CURS is one or more of CURS1, CURS2 or CURS3.

In one embodiment, the CURS is CURS1.

In one embodiment, the CURS is CURS2.

In one embodiment, the CURS is CURS3.

In one embodiment, there is provided a composition for promoting the expression of one or more curcuminoids in a cell, wherein the composition comprises:
a) one or more first vector components encoding DCS; and
b) one or more second vector components encoding a CURS.

In a further embodiment, the one or more second vector components encodes any one of the group comprising or consisting of CURS1, CURS2, or CURS3.

In a further embodiment, the one or more second vector components encodes any two of the group comprising or consisting of CURS1, CURS2, or CURS3.

In a further embodiment, the one or more second vector components encodes CURS1,

CURS2 and CURS3.

We have found that the combination of the enzymes, DCS and a CURS, in particular CURS3, promotes excellent expression of curcumin and other curcuminoids and their derivatives in Nicotiana tabacum plants and the plant cell line BY2. Surprisingly, the addition of transcription factors present in the flavonoid pathway, such as Del, Ros and MYB12, in combination with the enzymes DCS and a CURS, decreased the production of curcuminoids, presumably because the precursor compounds for the curcuminoids were diverted by the transcription factors to produce flavonoids.

In one embodiment there is provided, a composition for promoting the expression of one or more curcuminoids in a cell, wherein the composition comprises:
a) one or more first vector components encoding DCS and a CURS; and
b) one or more second vector components encoding one or more enzymes present in the curcuminoid pathway.

In a further embodiment, the one or more enzymes present in the curcuminoid pathway comprise one or more of the group comprising or consisting of a curcuminoid pathway hydroxylase, transferase, lyase, esterase and/or ligase.

In one embodiment, the one or more second vector components encode two or more enzymes present in the curcuminoid pathway.

In a further embodiment, the two or more enzymes present in the curcuminoid pathway comprise two or more of the group comprising or consisting of a curcuminoid pathway hydroxylase, transferase, lyase, esterase and/or ligase.

In one embodiment, the one or more second vector components encode three or more enzymes present in the curcuminoid pathway.

In a further embodiment, the three or more enzymes present in the curcuminoid pathway comprise three or more of the group comprising or consisting of a curcuminoid pathway hydroxylase, transferase, lyase, esterase and/or ligase.

In one embodiment, the one or more second vector components encode four or more enzymes present in the curcuminoid pathway.

In a further embodiment, the four or more enzymes present in the curcuminoid pathway comprise four or more of the group comprising or consisting of a curcuminoid pathway hydroxylase, transferase, lyase, esterase and/or ligase.

In one embodiment, the one or more second vector components encode five or more enzymes present in the curcuminoid pathway.

In a further embodiment, the five or more enzymes present in the curcuminoid pathway comprise five or more of the group comprising or consisting of a curcuminoid pathway hydroxylase, transferase, lyase, esterase and/or ligase.

In one embodiment, the one or more second vector components encode 6, 7, 8, 9, 10, 11, 12, 13, 14, 15, 16, 17, 18, 19, 20 or more enzymes present in the curcuminoid pathway.

In a further embodiment, the 6, 7, 8, 9, 10, 11, 12, 13, 14, 15, 16, 17, 18, 19, 20 or more enzymes present in the curcuminoid pathway comprise 6, 7, or 8 or more of the group comprising or consisting of a curcuminoid pathway hydroxylase, transferase, lyase, and/or ligase.

In one embodiment the hydroxylase is one or more of the group comprising or consisting of coumaroyl shikimate 3′-hydroxylase (C3′H) and/or cinnamate 4-hydroxylase (C4H).

In one embodiment, the transferase is one or more of the group comprising or consisting of hydroxycinnamoyltransferase (HCT), caffeoyl-CoA O-methyltransferase (CCoAOMT), catechol-O-methyltransferase (COMT), UDPG: glucosyltransferase, curcumin glucoside 1,6-glucosyltransferase, and/or flavonoid glucoside 1,6-glucosyltransferase.

In one embodiment, the lyase is one or more of the group comprising or consisting of phenylalanine ammonia lyase (PAL).

In one embodiment, the esterase is one or more of the group comprising or consisting of caffeoyl shikimate esterase (CSE).

In one embodiment, the ligase is one or more of the group comprising or consisting of 4-coumarate: CoA ligase (4CL).

PAL, C4H, 4CL, HCT, C3′H, CSE, COMT, and CCOAOMT are all enzymes associated with phenylpropanoid metabolism, which represents the first stage of the curcuminoid pathway. Increased expression of one or more of these enzymes leads to over expression of phenylpropanoids, which are then catalysed to form curcuminoids.

The skilled person will be aware that the maximum number of enzymes encoded by the second vector component will be dependent on the constraints of the vector construct chosen. E.g. the total sequence length that the vector construct can support.

The skilled person understands that enzymes present in the curcuminoid pathway may be isolated from a variety of different plant species. Thus, a plurality of different isoforms may exist for each enzyme described herein.

In selecting the combination of enzymes present in the curcuminoid pathway, the skilled person will use their knowledge of the curcuminoid pathway to select the appropriate enzymes to reach the desired curcuminoid compound.

Curcuminoids Obtainable from the Composition

As has already been disclosed, curcuminoids are found in the turmeric plant, Curcuma longa, among others. Curcuminoids are bioactive with many desirable properties, including anti-inflammatory and anticancer effects. Therefore, curcuminoids present a class of commercially attractive compounds.

As described herein, the composition promotes the expression of one or more curcuminoids.

In a further embodiment, the one or more curcuminoids comprise one or more plant-derived curcuminoids.

In one embodiment, the one or more curcuminoids comprise one or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the one or more curcuminoids is curcumin, or a derivative thereof.

In one embodiment, the one or more curcuminoids is bisdemethoxycurcumin, or a derivative thereof.

In one embodiment, the one or more curcuminoids is demethoxycurcumin, or a derivative thereof.

In one embodiment, the one or more curcuminoids is 5′-methoxycurcumin, or a derivative thereof.

In one embodiment, the one or more curcuminoids is dihydrocurcumin, or a derivative thereof.

In one embodiment, the one or more curcuminoids is cyclocurcumin, or a derivative thereof.

In one embodiment, the composition promotes the expression of two or more curcuminoids.

In a further embodiment, the two or more curcuminoids comprise two or more plant-derived curcuminoids.

In one embodiment, the two or more curcuminoids comprise two or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the two or more curcuminoids comprise curcumin, or a derivative thereof and at least one other curcuminoid.

In one embodiment, the two or more curcuminoids comprise bisdemethoxycurcumin, or a derivative thereof and at least one other curcuminoid.

In one embodiment, the two or more curcuminoids comprise demethoxycurcumin, or a derivative thereof and at least one other curcuminoid.

In one embodiment, the two or more curcuminoids comprise 5′-methoxycurcumin, or a derivative thereof and at least one other curcuminoid.

In one embodiment, the two or more curcuminoids comprise dihydrocurcumin, or a derivative thereof and at least one other curcuminoid.

In one embodiment, the two or more curcuminoids comprise cyclocurcumin, or a derivative thereof and at least one other curcuminoid.

In one embodiment, the composition promotes the expression of three or more curcuminoids.

In a further embodiment, the three or more curcuminoids comprise three or more plant-derived curcuminoids

In one embodiment, the three or more curcuminoids comprise three or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the three or more curcuminoids comprise curcumin, or a derivative thereof and at least two other curcuminoids.

In one embodiment, the three or more curcuminoids comprise bisdemethoxycurcumin, or a derivative thereof and at least two other curcuminoids.

In one embodiment, the three or more curcuminoids comprise demethoxycurcumin, or a derivative thereof and at least two other curcuminoids.

In one embodiment, the three or more curcuminoids comprise 5′-methoxycurcumin, or a derivative thereof and at least two other curcuminoids.

In one embodiment, the three or more curcuminoids comprise dihydrocurcumin, or a derivative thereof and at least two other curcuminoids.

In one embodiment, the three or more curcuminoids comprise cyclocurcumin, or a derivative thereof and at least two other curcuminoids.

In one embodiment, the composition promotes the expression of four or more curcuminoids.

In a further embodiment, the four or more curcuminoids comprise four or more plant-derived curcuminoids

In one embodiment, the four or more curcuminoids comprise four or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the four or more curcuminoids comprise curcumin, or a derivative thereof and at least three other curcuminoids.

In one embodiment, the four or more curcuminoids comprise bisdemethoxycurcumin, or a derivative thereof and at least three other curcuminoids.

In one embodiment, the four or more curcuminoids comprise demethoxycurcumin, or a derivative thereof and at least three other curcuminoids.

In one embodiment, the four or more curcuminoids comprise 5′-methoxycurcumin, or a derivative thereof and at least three other curcuminoids.

In one embodiment, the four or more curcuminoids comprise dihydrocurcumin, or a derivative thereof and at least three other curcuminoids.

In one embodiment, the four or more curcuminoids comprise cyclocurcumin, or a derivative thereof and at least three other curcuminoids.

In one embodiment, the composition promotes the expression of five or more curcuminoids.

In a further embodiment, the five or more curcuminoids comprise five or more plant-derived curcuminoids

In one embodiment, the five or more curcuminoids comprise five or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the five or more curcuminoids comprise curcumin, or a derivative thereof and at least four other curcuminoids.

In one embodiment, the five or more curcuminoids comprise bisdemethoxycurcumin, or a derivative thereof and at least four other curcuminoids.

In one embodiment, the five or more curcuminoids comprise demethoxycurcumin, or a derivative thereof and at least four other curcuminoids.

In one embodiment, the five or more curcuminoids comprise 5′-methoxycurcumin, or a derivative thereof and at least four other curcuminoids.

In one embodiment, the five or more curcuminoids comprise dihydrocurcumin, or a derivative thereof and at least four other curcuminoids.

In one embodiment, the five or more curcuminoids comprise cyclocurcumin, or a derivative thereof and at least four other curcuminoids.

In one embodiment, the composition promotes the expression of curcumin, bisdemethoxycurcumin, demethoxycurcumin, 5′-methoxycurcumin, dihydrocurcumin, and cyclocurcumin, or derivatives thereof.

The skilled person will understand that due to the nature of catalysis reactions, not all intermediate metabolites will be catalysed to form the next metabolite. The selected combinations of enzymes present in the curcuminoid pathway will promote the expression of particular curcuminoids. However, intermediate metabolites of the pathway may also be present. Therefore, a plurality of curcuminoids and intermediate metabolites may be expressed in the cell transfected with the composition described herein.

In certain embodiments, the curcuminoid obtained and the combination of enzymes present in the curcuminoid pathway are as recited in Table 1.

TABLE 1
provides non-limiting examples of combinations of enzymes
present in the curcuminoid pathway encoded by the vector
components as described herein, used to obtain curcuminoids.

| curcuminoid obtained | Genes encoded by the vector |
|---|---|
| curcumin | DCS & CURS1/CURS2/CURS3 |
| bisdemethoxycurcumin | DCS & CURS1/CURS2/CURS3 |
| demethoxycurcumin | DCS & CURS1/CURS2/CURS3 |

As is also exemplified in table 1, it is clear that there are combinations of enzymes present in the curcuminoid pathway common to multiple curcuminoids. Thus, a selected combination of enzymes present in the curcuminoid pathway encoded by the vector components, has the potential to promote the expression of a plurality of curcuminoids.

### Plant Products

As described herein, in one aspect there is provided a plant, plant part or plant cell transformed with the vector components according to any one of the composition embodiments described herein.

In one embodiment, the plant, plant part or plant cell is algae, moss or any plant, plant part or plant cell derived from the genus Nicotiana.

In a further embodiment, the plant, plant part or plant cell derived from the genus Nicotiana is one or more of the group comprising or consisting of Nicotiana tabacum, Nicotiana benthamiana, or BY-2 cells.

### Use

As described herein, in one aspect there is provided use of the composition according to any one of the composition embodiments described herein for promoting the expression of one or more curcuminoids in algae, moss, or any plant, plant part, or plant cells.

In one embodiment, the plant cells are a cell line derived from plants.

In one embodiment, the plant, plant part, or plant cells are derived from the genus Nicotiana.

In yet a further embodiment, the plant, plant part or plant cells derived from the genus Nicotiana are selected from the group comprising or consisting of Nicotiana tabacum, Nicotiana benthamiana, or BY-2 cells.

In an alternative embodiment, the expression of one or more curcuminoids is promoted in yeast cells and/or bacterial cells.

In one embodiment, the composition promotes the expression of one or more curcuminoids.

In a further embodiment, the one or more curcuminoids comprise one or more plant-derived curcuminoids.

In one embodiment, the one or more curcuminoids comprise one or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the one or more curcuminoids is curcumin, or a derivative thereof.

In one embodiment, the one or more curcuminoids is bisdemethoxycurcumin, or a derivative thereof.

In one embodiment, the one or more curcuminoids is demethoxycurcumin, or a derivative thereof.

In one embodiment, the one or more curcuminoids is 5′-methoxycurcumin, or a derivative thereof.

In one embodiment, the one or more curcuminoids is dihydrocurcumin, or a derivative thereof.

In one embodiment, the one or more curcuminoids is cyclocurcumin, or a derivative thereof.

In one embodiment, the composition promotes the expression of two or more curcuminoids.

In a further embodiment, the two or more curcuminoids comprise two or more plant-derived curcuminoids.

In one embodiment, the two or more curcuminoids comprise two or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the two or more curcuminoids comprise curcumin, or a derivative thereof and at least one other curcuminoid.

In one embodiment, the two or more curcuminoids comprise bisdemethoxycurcumin, or a derivative thereof and at least one other curcuminoid.

In one embodiment, the two or more curcuminoids comprise demethoxycurcumin, or a derivative thereof and at least one other curcuminoid.

In one embodiment, the two or more curcuminoids comprise 5′-methoxycurcumin, or a derivative thereof and at least one other curcuminoid.

In one embodiment, the two or more curcuminoids comprise dihydrocurcumin, or a derivative thereof and at least one other curcuminoid.

In one embodiment, the two or more curcuminoids comprise cyclocurcumin, or a derivative thereof and at least one other curcuminoid.

In one embodiment, the composition promotes the expression of three or more curcuminoids.

In a further embodiment, the three or more curcuminoids comprise three or more plant-derived curcuminoids

In one embodiment, the three or more curcuminoids comprise three or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the three or more curcuminoids comprise curcumin, or a derivative thereof and at least two other curcuminoids.

In one embodiment, the three or more curcuminoids comprise bisdemethoxycurcumin, or a derivative thereof and at least two other curcuminoids.

In one embodiment, the three or more curcuminoids comprise demethoxycurcumin, or a derivative thereof and at least two other curcuminoids.

In one embodiment, the three or more curcuminoids comprise 5′-methoxycurcumin, or a derivative thereof and at least two other curcuminoids.

In one embodiment, the three or more curcuminoids comprise dihydrocurcumin, or a derivative thereof and at least two other curcuminoids.

In one embodiment, the three or more curcuminoids comprise cyclocurcumin, or a derivative thereof and at least two other curcuminoids.

In one embodiment, the composition promotes the expression of four or more curcuminoids.

In a further embodiment, the four or more curcuminoids comprise four or more plant-derived curcuminoids

In one embodiment, the four or more curcuminoids comprise four or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the four or more curcuminoids comprise curcumin, or a derivative thereof and at least three other curcuminoids.

In one embodiment, the four or more curcuminoids comprise bisdemethoxycurcumin, or a derivative thereof and at least three other curcuminoids.

In one embodiment, the four or more curcuminoids comprise demethoxycurcumin, or a derivative thereof and at least three other curcuminoids.

In one embodiment, the four or more curcuminoids comprise 5′-methoxycurcumin, or a derivative thereof and at least three other curcuminoids.

In one embodiment, the four or more curcuminoids comprise dihydrocurcumin, or a derivative thereof and at least three other curcuminoids.

In one embodiment, the four or more curcuminoids comprise cyclocurcumin, or a derivative thereof and at least three other curcuminoids.

In one embodiment, the composition promotes the expression of five or more curcuminoids.

In a further embodiment, the five or more curcuminoids comprise five or more plant-derived curcuminoids

In one embodiment, the five or more curcuminoids comprise five or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the five or more curcuminoids comprise curcumin, or a derivative thereof and at least four other curcuminoids.

In one embodiment, the five or more curcuminoids comprise bisdemethoxycurcumin, or a derivative thereof and at least four other curcuminoids.

In one embodiment, the five or more curcuminoids comprise demethoxycurcumin, or a derivative thereof and at least four other curcuminoids.

In one embodiment, the five or more curcuminoids comprise 5′-methoxycurcumin, or a derivative thereof and at least four other curcuminoids.

In one embodiment, the five or more curcuminoids comprise dihydrocurcumin, or a derivative thereof and at least four other curcuminoids.

In one embodiment, the five or more curcuminoids comprise cyclocurcumin, or a derivative thereof and at least four other curcuminoids.

In one embodiment, the composition promotes the expression of curcumin, bisdemethoxycurcumin, demethoxycurcumin, 5′-methoxycurcumin, dihydrocurcumin, and cyclocurcumin, or derivatives thereof.

### Method

As described herein, in one aspect there is provided a method of promoting the expression of one or more curcuminoids in a cell, comprising the step of:
a) introducing the composition according to any one of the composition embodiments described herein into a cell.

In one embodiment, the method further comprises:
b) extracting the one or more curcuminoids from the cell to produce an extract.

In an additional embodiment, the method further comprises:
c) detecting the one or more curcuminoids in the extract.

In an additional embodiment, the method further comprises:
d) purifying the one or more curcuminoids from the extract.

In one embodiment, the method further comprises one or more of:
b) extracting the one or more curcuminoids from the cell to produce an extract;
c) detecting the one or more curcuminoids in the extract; and
d) purifying the one or more curcuminoids from the extract.

In one embodiment, the method further comprises:
b) extracting the one or more curcuminoids from the plant to produce an extract;
c) detecting the one or more curcuminoids in the extract; and
d) purifying the one or more curcuminoids from the extract.

In a further embodiment, the one or more curcuminoids are purified using Flash and preparative MPLC, HPTLC or LC/MS.

In one embodiment, the cell is a plant cell, yeast cell and/or bacteria cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In one embodiment, the composition promotes the expression of one or more curcuminoids.

In a further embodiment, the one or more curcuminoids comprise one or more plant-derived curcuminoids.

In one embodiment, the one or more curcuminoids comprise one or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the one or more curcuminoids is curcumin, or a derivative thereof.

In one embodiment, the one or more curcuminoids is bisdemethoxycurcumin, or a derivative thereof.

In one embodiment, the one or more curcuminoids is demethoxycurcumin, or a derivative thereof.

In one embodiment, the one or more curcuminoids is 5′-methoxycurcumin, or a derivative thereof.

In one embodiment, the one or more curcuminoids is dihydrocurcumin, or a derivative thereof.

In one embodiment, the one or more curcuminoids is cyclocurcumin, or a derivative thereof.

In one embodiment, the composition promotes the expression of two or more curcuminoids.

In a further embodiment, the two or more curcuminoids comprise two or more plant-derived curcuminoids.

In one embodiment, the two or more curcuminoids comprise two or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the two or more curcuminoids comprise curcumin, or a derivative thereof and at least one other curcuminoid.

In one embodiment, the two or more curcuminoids comprise bisdemethoxycurcumin, or a derivative thereof and at least one other curcuminoid.

In one embodiment, the two or more curcuminoids comprise demethoxycurcumin, or a derivative thereof and at least one other curcuminoid.

In one embodiment, the two or more curcuminoids comprise 5′-methoxycurcumin, or a derivative thereof and at least one other curcuminoid.

In one embodiment, the two or more curcuminoids comprise dihydrocurcumin, or a derivative thereof and at least one other curcuminoid.

In one embodiment, the two or more curcuminoids comprise cyclocurcumin, or a derivative thereof and at least one other curcuminoid.

In one embodiment, the composition promotes the expression of three or more curcuminoids.

In a further embodiment, the three or more curcuminoids comprise three or more plant-derived curcuminoids

In one embodiment, the three or more curcuminoids comprise three or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the three or more curcuminoids comprise curcumin, or a derivative thereof and at least two other curcuminoids.

In one embodiment, the three or more curcuminoids comprise bisdemethoxycurcumin, or a derivative thereof and at least two other curcuminoids.

In one embodiment, the three or more curcuminoids comprise demethoxycurcumin, or a derivative thereof and at least two other curcuminoids.

In one embodiment, the three or more curcuminoids comprise 5′-methoxycurcumin, or a derivative thereof and at least two other curcuminoids.

In one embodiment, the three or more curcuminoids comprise dihydrocurcumin, or a derivative thereof and at least two other curcuminoids.

In one embodiment, the three or more curcuminoids comprise cyclocurcumin, or a derivative thereof and at least two other curcuminoids.

In one embodiment, the composition promotes the expression of four or more curcuminoids.

In a further embodiment, the four or more curcuminoids comprise four or more plant-derived curcuminoids

In one embodiment, the four or more curcuminoids comprise four or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the four or more curcuminoids comprise curcumin, or a derivative thereof and at least three other curcuminoids.

In one embodiment, the four or more curcuminoids comprise bisdemethoxycurcumin, or a derivative thereof and at least three other curcuminoids.

In one embodiment, the four or more curcuminoids comprise demethoxycurcumin, or a derivative thereof and at least three other curcuminoids.

In one embodiment, the four or more curcuminoids comprise 5′-methoxycurcumin, or a derivative thereof and at least three other curcuminoids.

In one embodiment, the four or more curcuminoids comprise dihydrocurcumin, or a derivative thereof and at least three other curcuminoids.

In one embodiment, the four or more curcuminoids comprise cyclocurcumin, or a derivative thereof and at least three other curcuminoids.

In one embodiment, the composition promotes the expression of five or more curcuminoids.

In a further embodiment, the five or more curcuminoids comprise five or more plant-derived curcuminoids

In one embodiment, the five or more curcuminoids comprise five or more of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the five or more curcuminoids comprise curcumin, or a derivative thereof and at least four other curcuminoids.

In one embodiment, the five or more curcuminoids comprise bisdemethoxycurcumin, or a derivative thereof and at least four other curcuminoids.

In one embodiment, the five or more curcuminoids comprise demethoxycurcumin, or a derivative thereof and at least four other curcuminoids.

In one embodiment, the five or more curcuminoids comprise 5′-methoxycurcumin, or a derivative thereof and at least four other curcuminoids.

In one embodiment, the five or more curcuminoids comprise dihydrocurcumin, or a derivative thereof and at least four other curcuminoids.

In one embodiment, the five or more curcuminoids comprise cyclocurcumin, or a derivative thereof and at least four other curcuminoids.

In one embodiment, the composition promotes the expression of curcumin, bisdemethoxycurcumin, demethoxycurcumin, 5′-methoxycurcumin, dihydrocurcumin, and cyclocurcumin, or derivatives thereof.

### Products

As described herein, in one aspect there is provided a curcuminoid obtainable from the method according to any one of the method embodiments described herein.

In one embodiment, the curcuminoid is a plant-derived curcuminoid.

In one embodiment, the curcuminoid is any one of the group comprising or consisting of curcumin, bisdemethoxycurcumin, demethoxycurcumin, 5′-methoxycurcumin, dihydrocurcumin, and cyclocurcumin, or derivatives thereof.

In one embodiment, the curcuminoid is curcumin or a derivative thereof.

In one embodiment, the curcuminoid is bisdemethoxycurcumin or a derivative thereof.

In one embodiment, the curcuminoid is demethoxycurcumin or a derivative thereof.

In one embodiment, the curcuminoid is 5′-methoxycurcumin or a derivative thereof.

In one embodiment, the curcuminoid is dihydrocurcumin or a derivative thereof.

In one embodiment, the curcuminoid is cyclocurcumin or a derivative thereof.

The expressed curcuminoids, may or may not be endogenously present in the cell, cell line, yeast, bacteria, algae, moss, or plant in which the composition of the invention is used. It will be understood by the skilled person that where a particular curcuminoid is not endogenously produced, there is considered to be an increase in this curcuminoid if it can be detected after the cell, cell line, yeast, bacteria, algae, moss or plant has been transfected with the composition of the invention.

In situations where particular curcuminoids are endogenously expressed in the cell, cell line, yeast, bacteria, algae, moss, or plant before the composition of the invention is used, a 1-fold, 2-fold, 3-fold, 4-fold, 5-fold, 10-fold, 20-fold, 40-fold, 60-fold, 80-fold, 100-fold, 150-fold, 200-fold, 300-fold, 400-fold, 600-fold, 800-fold, 1000-fold, or more increase in that particular curcuminoid may be observed.

The total amount of curcuminoids present in a cell, cell line, yeast, bacteria, algae, moss, or plant in which the composition of the invention is used may be increased by 1-fold, 2-fold, 3-fold, 4-fold, 5-fold, 10-fold, 20-fold, 40-fold, 60-fold, 80-fold, 100-fold, 150-fold, 200-fold, 250-fold, or more.

The various aspects and/or embodiments described herein are presented only to assist in understanding and teaching the claimed features. These aspects and/or embodiments are provided as a representative sample of aspects and/or embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

The invention will now be described with reference to the following non-limiting examples.

### EXAMPLES

### Example 1: Promoting the Expression of Curcumin in Nicotiana tabacum

Nicotiana tabacum plants were selected for transfection with plasmids. The three plasmids generated comprised DCS & CURS3, DEL/ROS, and MYB12.

An Agrobacterium culture (Agrobacterium tumefaciens) was used to deliver the genetic material into the Nicotiana tabacum cells. The three plasmids containing the genes of interest were introduced into separate Agrobacterium cultures and cultured overnight to reach specific density.

Once the specific density was reached (OD600 of ~1-2), it's resuspended at a density of OD600 0.2 . . . . Infiltration solutions were prepared for each of the three plasmid types separately (DCS & CURS3, & DEL/ROS, and MYB12).

The three infiltration solutions were then individually infiltrated into the leaves of the Nicotiana tabacum plants, using a syringe or vacuum infiltration.

Separate plants were used for each set (T1, T2 and T3). For T1 DCS & CURS3 plasmid was used. For T2, DCS & CURS3 and Del/Ros were used. For T3, DCS & CURS3 and MYB12 were used.

Following infiltration, the plants were incubated under controlled conditions (22° C., ~250 μmol/m2/s (light), 70% humidity and 16 hr day/night cycle.) in a growth chamber for 7 days. Following this, the infiltrated leaves were removed from the plant and frozen instantly.

The dried leaves were then grounded and extraction was preformed using methanol. Extracts were then filtered and placed in vials to be analysed by HPTLC. Stationary Phase; Merck, HPTLC Silica gel 60 F₂₅₄, Mobile phase; dichloromethane-methanol 9.7:0.3 (v/v). After separation, the plates were derivatized with 10% H2SO4 in methanol and then visualized by normal light or UV lamp.

### Results

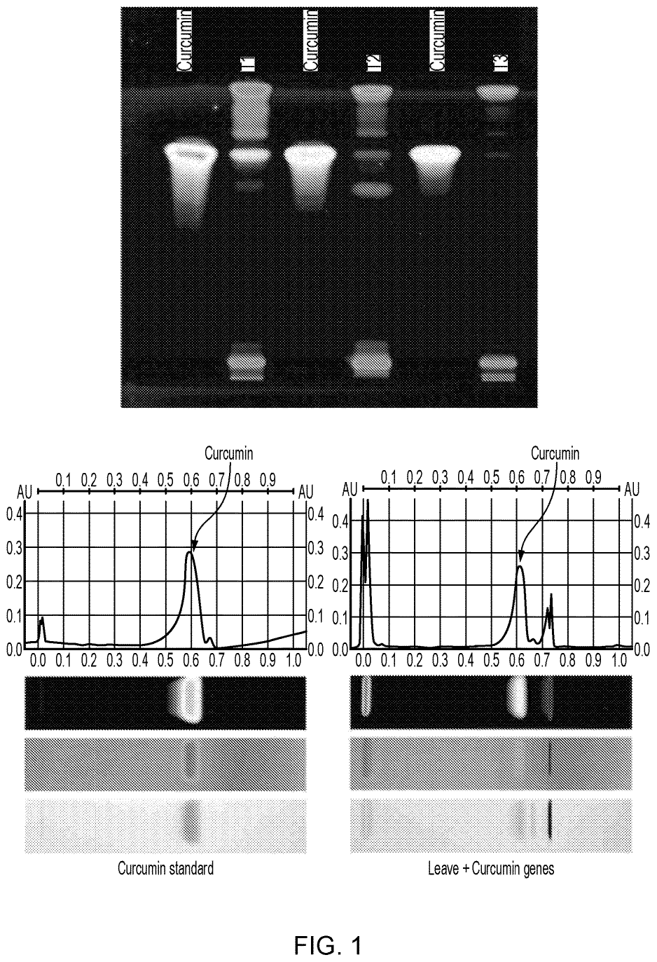

When compared to a curcumin standard (FIG. 1) it was clear that the expression of curcumin was promoted, demonstrated by the Rf value matching that of the curcuminoid compound standard. In addition, both the analysed sample and the curcuminoid compound standard possess yellow fluorescence under UV light (366 nm). Surprisingly, addition of the transcription factors DEL/ROS or MYB12 reduced the amount of curcumin expressed, compared to the combination of DCS & CURS3 alone. Additional peaks seen in the HPTLC suggest that other curcuminoid compounds were also promoted.

1. A composition for promoting the expression of one or more curcuminoids, wherein the composition comprises:
a) one or more first vector components encoding one or more enzymes present in the curcuminoid pathway; and optionally
b) one or more second vector components encoding one or more enzymes present in the curcuminoid pathway, different to the one or more enzymes present in the curcuminoid pathway encoded by the first vector component.

2. The composition according to claim 1, wherein the one or more enzymes present in the curcuminoid pathway comprise DCS.

3. The composition according to claim 1 or claim 2, wherein the one or more enzymes present in the curcuminoid pathway comprise a CURS.

4. The composition according to any one of claims 1 to 3, wherein the composition comprises:
a) one or more first vector components encoding DCS; and
b) one or more second vector components encoding a CURS.

5. The composition according to claim 3 or claim 4, wherein the CURS is one or more of CURS1, CURS2 or CURS3.

6. The composition according to claim 3 or claim 4, wherein the CURS is CURS1.

7. The composition according to claim 3 or claim 4, wherein the CURS is CURS2.

8. The composition according to claim 3 or claim 4, wherein the CURS is CURS3.

9. A plant, plant part or plant cell transformed with the vector components according to the composition of any one of claims 1-8.

10. The plant, plant part or plant cell according to claim 9, wherein the plant, plant part or plant cell is algae, moss or any plant, plant part or plant cell derived from the genus Nicotiana.

11. The plant, plant part or plant cell according to claim 10, wherein the plant, plant part of plant cell derived from the genus Nicotiana is selected from the group comprising Nicotiana tabacum, Nicotiana benthamiana, or BY-2 cells.

12. Use of the composition according to any one of claims 1-8 for promoting the expression of one or more curcuminoids in algae, moss, or any plant, plant part, or plant cells.

13. Use of the composition according claim 12, wherein the plant cells are a cell line derived from plants.

14. Use of the composition according to claim 13, wherein the plant, plant part, or plant cells are derived from the genus Nicotiana.

15. Use of the composition according to claim 14, wherein the plant, plant part or plant cells derived from the genus Nicotiana are selected from the group comprising Nicotiana tabacum, Nicotiana benthamiana, or BY-2 cells.

16. A method of promoting the expression of one or more curcuminoids in a cell, comprising the step of:
a) introducing the composition according to any one of claims 1-8 into a cell.

17. The method of claim 16, wherein the method further comprises:
b) extracting the one or more curcuminoids from the cell to produce an extract;
c) detecting the one or more curcuminoids in the extract; and
d) purifying the one or more curcuminoids from the extract.

18. The method according to claim 17, wherein the one or more curcuminoids are purified using Flash and preparative MPLC, HPTLC or LC/MS.

19. The method according to any one or claims 16-18, wherein the cell is a plant cell.

20. The method according to claim 19, wherein the plant cell is an algae cell, moss cell, or any cell or cell line derived from the genus Nicotiana.

21. The method according to claim 20, wherein the cell or cell line derived from the genus Nicotiana is selected from the group comprising Nicotiana tabacum, Nicotiana benthamiana, or BY-2 cells.

22. A curcuminoid obtainable from the method according to any one of claims 14-19.


SEQUENCE LISTING (2 sequences)

SEQ ID NO: 1 - DNA, 14042 bp, synthetic construct
  source 1..14042: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggaagcgaacggctaccgcataactcacagcgccgacgggccggcgacgatcttggccatcggcaccgccaaccccaccaacgtcgtcgatcagaacgcttatcccgacttctatttccgggtcaccaactccgagtatctgcaggaactcaaagccaagtttaggcgcatctgtgagaaagcggccatcaggaagaggcacttgtacttgactgaggagattttgcgggagaatcctagcttgctggctcccatggcgccgtcgttcgacgcgcggcaggcgatcgtggtggaggcggtgccgaagctggcgaaggaggcggcggagaaggcgatcaaggagtggggccgccccaaatcggacatcacgcacctcgtcttctgctccgcgagcggaatcgacatgcccggctccgacctgcagcttctcaagctgctcgggctcccgccgagcgtcaatcgcgtcatgctctacaacgtcgggtgccacgccggtggcaccgccctccgcgtcgccaaggacctcgcggagaacaaccgcggcgcgcgggtgctcgccgtctgctccgaggtcaccgtgctctcctaccgcggcccccaccccgcccacatcgagagcctcttcgtccaagctctgtttggcgacggcgccgccgcgctcgtggtcgggtccgaccccgtcgatggcgtcgagcgccccatcttcgaaatcgcctcggcatcccaagtgatgcttccggagagcgcagaggcggtgggcggccacctccgcgaaattgggctgaccttccacctcaagagccagcttccgtcgatcatcgcgagcaacatcgagcagagcctgacgactgcgtgctcgccgctggggctgtcggactggaaccagctgttctgggcggttcaccccggcggccgagcgatcctggaccaggtggaggcgcggctcggactggagaaggaccggctcgccgcgacgcggcacgtactcagcgagtacggcaacatgcagagcgccacggtgctgttcatcctggacgagatgcggaaccgctcggctgcggagggccacgccaccaccggcgaggggctcgactggggcgtgctgttgggcttcggcccgggactctccatcgagaccgtcgtcctccatagttgcagactgaactagcccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggcagcctgcaggcgatgcgcagggcacagcgggctcaaggcccggccaccatcatggctgtcggcacctccaacccgcctaacctctacgagcagacctcctaccccgacttctatttccgcgtcaccaactccgaccacaagcacgcgctcaagaacaaattccgtgttatctgtgagaagacgaaggtgaaaagacggtacttgcacttgacggaggagatcctgaagcagaggcccaagctctgctcctacatggagccctccttcgacgatcggcaggacatcgtggtggaggagataccaaagctggcgaaggaggcggcggagaaggcgatcaaggagtggggccgccccaagtcggagatcacccacttggtgttctgctccatcagcggtatcgacatgcccggcgccgattaccgcctcgccaccctcctcggcctccccctgtccgtcaaccgcctcatgctctacagccaggcctgccacatgggcgcgcagatgctgcgcatagccaaggacctcgcggagaacaaccggggcgcgcgcgtcctggccgtctcctgcgaaatcaccgtgctcagcttccgcggtccggacgcgggcgacttcgaggccctcgcgtgtcaggccggcttcggcgacggtgccgctgccgtcgtcgtcggggccgaccccctcccgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacaggggcgtcgagaggcccatctacgagatcgcggcggcgatgcaggaaacggtgccggagagcgagagggcggtggggggccacctgagggaaatcggctggaccttccacttcttcaaccagcttccgaagctgatcgcggaaaacatcgagggcagcctggcgcgggcgttcaagccgctggggatcagcgagtggaacgacgtgttctgggtggcgcacccggggaactggggcatcatggacgccatcgagaccaagctggggctggaacaggggaagctcgccacggcgcgccacgtcttcagcgagtacggaaacatgcagagcgccaccgtgtacttcgtgatggacgaggtgaggaagcggtcggcggcggaggggcgggccaccaccggcgaaggcctggagtggggagtgctgtttgggtttggcccaggcctcaccatagaaactgtcgtgctacgcagtgtaccattaccgtagcccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 2 - DNA, 8635 bp, synthetic construct
  source 1..8635: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagccaaaatgcatgccaccggcatccagaaccagaagatcgtgcccgagaacctgcgcaagcagctggccattgcggtgcgctctatccagtggtcgtacgcgatcttctggagcaacagcgtggcgcagccgggagtgctggagtggggcgacggcttctacaacggcgacatcaagacgcgcaagaccgtgcagagcgtggagctgaaccaggaccagctgggcctgcagcgcagcgaccagctgcgcgagctgtacgagagcctgagcctgggcgagaccaacacgcaggcgaagcgccccaccgcggccctgagccccgaggacctgaccgacgccgagtggttcttcctggtgtgcatgagcttcatcttcaacatcggccagggcctgccgggccgcaccctggcccgcaaccaggctgtgtggctgtgcaacgcccaccgcgccgacaccaaggtgttctcccgcagcctgctggccaagtccgcgtccatccagaccgtggtgtgcttcccctacagcgagggcgtggtcgagctgggcgccacggagctggtgcccgaggacctgaacctgatccagcacatcaagacctcgttcctggacagccccgccaccgtgcccaagatcccgaactacgtgagcaacagcatcaccaataacaatgacctgatctgcgaggcgctggagcacgccaacatccccgagaacgacctggaccagctgctgaactgccccgacaccaacatctgctcccccgacaactctctggacgacttcgccgacaacctgctgatcgacgagagcaacctggcggagggcatcaacggggaggtgccccagacgcagagctggcccttcatggacgacgccatcagcaactgtctgaacagcagcatgaacagcagcgactgcatcagccagacccacgagaacctggagagcttcgcgccgctgagcgacggcaagggcccccccgagacgaacaactgcatgcacagcacccagaagtgcaaccagcagatcgagaacaccggcgtgcagggcgacgaggtgcactaccagggcgtgctgagcaacctgctgaagtcgtcccaccagctggtgctgggcccgtacttccgcaacggcaaccgcgagagctcgttcgtgagctggaacaaggacggcagctcgggcacccacgtgccgcgctcgggcacctcccagcgcttcctgaagaaggtgctgttcgaggtggcccgcatgcacgagaactcgcgcctggacgcgggcaagcagaagggcaacagcgactgcctcgccaagcccaccgctgacgagatcgaccgcaaccacgtgctgtccgagcgcaagcgccgcgagaagatcaacgagcgcttcatgatcctggcgagcctggtccccagcggcggcaaggtggacaaggtgagcatcctggaccacaccattgactacctgcgcggcctggagcgcaaggtggacgagctggagagcaacaagatggtgaagggccgcggccgcgagagcaccaccaagaccaagctgcacgacgccatcgagcgcacgagcgacaactacggcgcgacccgcacctccaacgtgaagaagcccctgaccaacaagcgcaaggcctccgacaccgacaagatcggcgcggtgaacagccgcggccgcctgaaggactcgctgacggacaacatcaccgtgaacatcaccaacaaggacgtgctgatcgtggtgacatgcagcagcaaggagttcgtcctgctggaggtgatggaggcggtgcgccgcctgtccctggacagcgagaccgtgcagagctccaaccgggacggcatgatcagcatcaccatcaaggctaagtgcaagggcctgaaggtggcctccgccagcgtgatcaagcaggcgctgcagaaggtgaccatgaagagcctgctggcgatccaccccaccgaggcgcgccacaagcagaagattgtggcccccgtgaagcagaccctgaacttcgacctgctgaagctggcgggcgacgtggagtccaacccgggccccatggagaagaactgccgcggcgtgcgcaagggcacctggaccaaggaggaggacaccctgctgcgccagtgcatcgaggagtacggcgagggcaagtggcaccaggtgccccaccgcgccggcctgaaccgctgccgcaagtcctgccgcctgcgctggctgaactacctgcgcccgaacatcaagcgcggccgcttcagccgcgacgaggtggacctgatcgtgcgcctgcacaagctgctgggcaacaagtggagcctgatcgccggccgcatccccggccgcaccgcgaacgacgtgaagaacttctggaacacccacgtgggcaagaacctgggcgaggacggcgagcgctgccgcaagaacgtgatgaacaccaagaccattaagctgaccaacatcgtgcgcccgcgcgcgcgcaccttcaccggcctgcacgtgacctggccccgcgaggtgggcaagacggacgagttctccaacgtgcgcctgaccacggacgagatccccgactgcgagaagcagacgcagttctacaacgacgtggcgtccccgcaggacgaggtggaggactgcatccagtggtggtcgaagctgctggagaccaccgaggacggcgagctgggcaacctgttcgaggaggcgcagcagatcggcaactaaccggtgcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagagtcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgacacaagaatccctgttacttctcgaccgtattgattcggatgattcctacgcgagcctgcggaacgaccaggaattctgggaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgataaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc